(19) United States
(12) Patent Application Publication
(10) Pub. No.: US 2025/0318797 A1
(43) Pub. Date: Oct. 16, 2025

(54) SYSTEMS, METHODS, AND DEVICES FOR X-RAY IMAGING

(71) Applicant: SHANGHAI UNITED IMAGING HEALTHCARE CO., LTD., Shanghai (CN)

(72) Inventors: Fan ZHAO, Shanghai (CN); Guoping ZHU, Shanghai (CN); Bin CAO, Shanghai (CN)

(73) Assignee: SHANGHAI UNITED IMAGING HEALTHCARE CO., LTD., Shanghai (CN)

(21) Appl. No.: 19/245,380

(22) Filed: Jun. 22, 2025

Related U.S. Application Data
(63) Continuation of application No. PCT/CN2023/141244, filed on Dec. 22, 2023.

(30) Foreign Application Priority Data
Dec. 22, 2022 (CN) 202211659619.4

(51) Int. Cl. A61B 6/58; A61B 6/00; A61B 6/40

(52) U.S. Cl. CPC A61B 6/583; A61B 6/52; A61B 6/54; A61B 6/40; A61B 6/4441

(57) ABSTRACT

Embodiments of the present disclosure may provide a system, a method, and a device for X-ray imaging. The method may include: obtaining a grayscale of a current frame of an object, wherein the current frame is obtained by performing X-ray imaging on the object based on current performing parameters; determining an updated equivalent phantom thickness of the object based on the grayscale of the current frame, the current performing parameters, and a pre-stored correspondence, wherein the pre-stored correspondence reflects dose values corresponding to a plurality of equivalent phantom thicknesses and a plurality of performing parameters; and determining target performing parameters of a next frame of the object based on the updated equivalent phantom thickness, a target grayscale, and the pre-stored correspondence.

### CROSS-REFERENCE TO RELATED APPLICATION

This application is a continuation of International Patent Application No. PCT/CN2023/141244, filed on Dec. 22, 2023, which claims priority of Chinese Patent Application No. 202211659619.4, filed on Dec. 22, 2022, the entire contents of which are incorporated herein by reference.

### TECHNICAL FIELD

The present disclosure relates to the field of medical imaging, and in particular, to systems, methods, and devices for X-ray imaging.

### BACKGROUND

Automatic Brightness Stabilization (ABS) technology is a control method applied to maintain the consistency of image brightness in systems for medical X-ray imaging. With a certain dose of initial X-rays, since different objects (usually patients) to be detected have different abilities to block X-rays, attenuation degrees of X-rays transmitted through the objects to be detected are also different. The higher the attenuation degree of the X-rays, the darker the image brightness; and the lower the attenuation degree of the X-rays, the brighter the image brightness. Both a high attenuation degree and a low attenuation degree result in an unclear image due to a lack of an optimal brightness. In order to optimize the image brightness, it is necessary to adjust the dose of the X-rays so that the image grayscale meets a requirement.

Accordingly, there is a need to provide systems, methods, and devices for X-ray imaging to optimize X-ray imaging and to shorten a stabilization time of a control method for a system for X-ray imaging.

### SUMMARY

One or more embodiments of the present disclosure may provide a system for X-ray imaging, comprising: a storage device storing a set of instructions; and at least one processor in communication with the storage device. When executing the set of instructions, the at least one processor may be directed to cause the system to perform operations including: obtaining a grayscale of a current frame of an object, wherein the current frame is obtained by performing X-ray imaging on the object based on current performing parameters; determining an updated equivalent phantom thickness of the object based on the grayscale of the current frame, the current performing parameters, and a pre-stored correspondence, wherein the pre-stored correspondence reflects dose values corresponding to a plurality of equivalent phantom thicknesses and a plurality of performing parameters; and determining target performing parameters of a next frame of the object based on the updated equivalent phantom thickness, a target grayscale, and the pre-stored correspondence.

One or more embodiments of the present disclosure may provide a method for X-ray imaging. The method may include: obtaining a grayscale of a current frame of an object, wherein the current frame is obtained by performing X-ray imaging on the object based on current performing parameters; determining an updated equivalent phantom thickness of the object based on the grayscale of the current frame, the current performing parameters, and a pre-stored correspondence, wherein the pre-stored correspondence reflects dose values corresponding to a plurality of equivalent phantom thicknesses and a plurality of performing parameters; and determining target performing parameters of a next frame of the object based on the updated equivalent phantom thickness, a target grayscale, and the pre-stored correspondence.

One or more embodiments of the present disclosure may provide a non-transitory computer-readable storage medium storing computer instructions. After reading the computer instructions in the storage medium, a computer may execute the method for X-ray imaging described above.

The control method of the system for X-ray imaging provided by the embodiments of the present disclosure may update an equivalent phantom thickness based on a pre-stored correspondence and determine performing parameters of an X-ray according to the correspondence, thereby realizing the adjustment of image brightness. By performing parameters based on the pre-obtained correspondence, a speed of parameter adjustment can be increased and a stabilization time corresponding to a process for automatic brightness adjustment can be shortened.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary system for X-ray imaging according to some embodiments of the present disclosure;

FIG. 2 is a flowchart illustrating an exemplary process for X-ray imaging according to some embodiments of the present disclosure;

FIG. 3 is a flowchart illustrating an exemplary process for determining an updated equivalent phantom thickness according to some embodiments of the present disclosure;

FIG. 4 is a flowchart illustrating an exemplary process for determining a correspondence according to some embodiments of the present disclosure;

FIG. 5 is a flowchart illustrating another exemplary process for determining a correspondence according to some embodiments of the present disclosure;

FIG. 6 is a flowchart illustrating an exemplary control process for a system for X-ray imaging according to some embodiments of the present disclosure;

FIG. 7 is a flowchart illustrating an exemplary process for obtaining current performing parameters of a current frame according to some embodiments of the present disclosure;

FIG. 8 is a flowchart illustrating operation S602 according to some embodiments of the present disclosure;

FIG. 9 is a flowchart illustrating another exemplary control process for a system for X-ray imaging according to some embodiments of the present disclosure, respectively;

FIG. 10 is a flowchart illustrating another exemplary control process for a system for X-ray imaging according to some embodiments of the present disclosure, respectively;

FIG. 11 is a flowchart illustrating another exemplary control process for a system for X-ray imaging according to some embodiments of the present disclosure, respectively;

FIG. 12 is a flowchart illustrating another exemplary control process for a system for X-ray imaging according to some embodiments of the present disclosure, respectively;

FIG. 13 is a flowchart illustrating another exemplary control process for a system for X-ray imaging according to some embodiments of the present disclosure, respectively;

FIG. 14 is a flowchart illustrating another exemplary control process for a system for X-ray imaging according to some embodiments of the present disclosure, respectively;

FIG. 15 is a block diagram illustrating a control device for a system for X-ray imaging according to some embodiments of the present disclosure;

FIG. 16 is a block diagram illustrating an updating module according to some embodiments of the present disclosure;

FIG. 17 is a block diagram illustrating another control device for a system for X-ray imaging according to some embodiments of the present disclosure;

FIG. 18 is a block diagram illustrating another control device for a system for X-ray imaging according to some embodiments of the present disclosure; and

FIG. 19 is a schematic diagram illustrating a structure of an electronic device according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. Unless obviously obtained from the context or the context illustrates otherwise, the same numeral in the drawings refers to the same structure or operation.

It will be understood that the terms “system,” “device,” “unit,” and/or “module” used herein are one method to distinguish different components, elements, parts, sections, or assemblies of different levels in ascending order. However, the terms may be displaced by other expressions if they may achieve the same purpose.

As used herein, the singular forms “a,” “an,” and “the” may be intended to include the plural forms as well, unless the context clearly indicates otherwise. It will be further understood that the terms “include” and/or “comprise,” when used in this disclosure, specify the presence of operations and/or elements, but do not exclude the presence or addition of one or more other operations and/or elements thereof.

The flowcharts used in the present disclosure illustrate operations that systems implement according to some embodiments of the present disclosure. It is to be expressly understood, the operations of the flowcharts may be implemented not in order. Conversely, the operations may be implemented in an inverted order, or simultaneously. Moreover, one or more other operations may be added to the flowcharts. One or more operations may be removed from the flowcharts.

In order to optimize the image brightness of an X-ray image, it is necessary to adjust the dose of the X-rays. Specifically, if the image brightness is relatively dark, the dose of the X-rays may be increased; and if the image brightness is relatively bright, the dose of the X-rays may be decreased. In the process of brightness adjustment, a duration from starting imaging an object to obtaining a stable perspective image that has an optimal brightness may be designated as a stabilization time of an ABS perspective. The stabilization time is one of key performance indicators for measuring the ABS perspective. Because the perspective image is unstable, the dose of the X-rays received by the object to be detected during the stabilization time of the ABS perspective is useless. Therefore, the shorter the stabilization time of the ABS perspective, the better it is for the object to be detected.

An X-ray bulb tube is a device capable of generating X-rays. The dose of the X-rays may be adjusted by adjusting a perspective voltage and a perspective current. The perspective voltage refers to a voltage between a cathode and an anode of the X-ray bulb tube, which is usually within a range of 40-150 kV. The perspective current refers to a current between the cathode and the anode of the X-ray bulb tube, which is usually within a range of 10-30 mA. In order to achieve a perspective image with a moderate brightness, both the perspective voltage and the perspective current may be adjusted during an adjustment time of the ABS perspective. The perspective voltage may be adjusted immediately by changing an input signal of a digital-to-analog converter (ADC) in an X-ray high voltage generator (HVG) and then performing boost rectification through the high-voltage oil tank. A filament current supplied to the X-ray bulb tube by the X-ray HVG may be set to heat the filament to change a filament temperature, thus the perspective current may be adjusted by changing a count of electrons emitted by the filament to an anode target surface of the bulb tube according to the filament temperature. Because the change in the filament temperature due to the change in the filament current of the bulb tube is a slow process, which takes about tens to hundreds of milliseconds, resulting in a long time to adjust the perspective current.

Related technologies have used one or more proportional proportion-integral integration-differential differentiation (PID) controllers to realize the adjustment of the perspective voltage and the perspective current. A perspective voltage and a perspective current before the adjustment and an initial brightness of an image obtained by X-ray fluoroscopy of the object to be detected may be obtained and designated as control parameters to be input into the PID controllers. A difference between the initial brightness and a preset brightness may be used to calculate adjustment amounts of the perspective voltage and perspective current, thereby realizing a purpose of adjusting the image brightness. If the adjustment amount of the perspective current is too large, the perspective current may oscillate, resulting in a longer time required for the image to stabilize; conversely, if the adjustment amount of the perspective current is too small, repeatedly adjustments may be required. Since each adjustment of the perspective current takes a long time, an overall adjustment time may be too long. Therefore, a time for adjusting the perspective current using the PID control method tends to be relatively long.

In view of the foregoing, some embodiments of the present disclosure disclose a system and a method for X-ray imaging. The system and the method may use a preset correspondence between phantom thicknesses and X-ray performing parameters to update a phantom thickness of an object, and determine target performing parameters of a next frame of the object quickly based on the preset correspondence, thereby realizing a rapid adjustment of the image brightness.

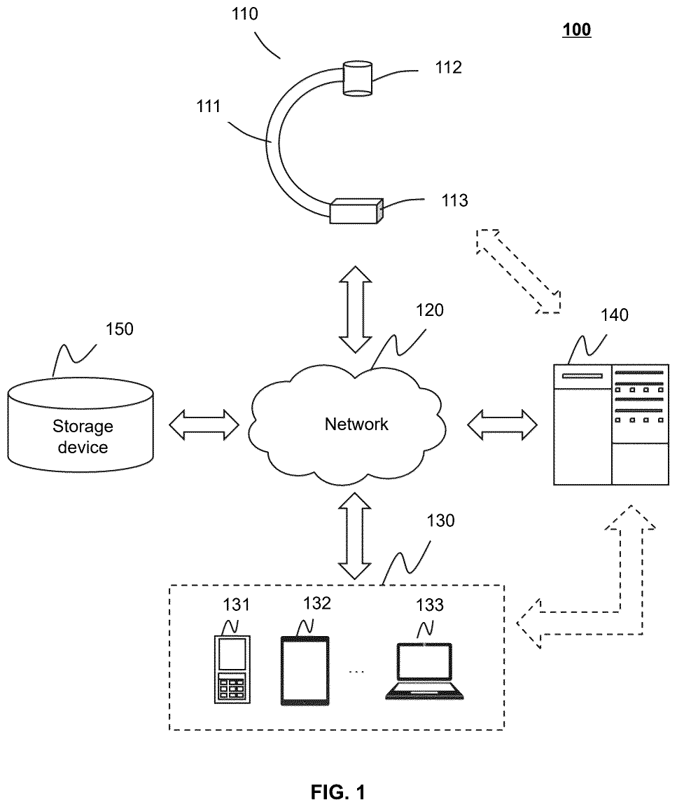

FIG. 1 is a schematic diagram illustrating an exemplary system 100 for X-ray imaging according to some embodiments of the present disclosure. As shown in FIG. 1, the system 100 for X-ray imaging may include a device 110 for X-ray imaging, a network 120, one or more terminals 130, a processing device 140, and a storage device 150. Connections between components in the system 100 for X-ray imaging may be variable. For example, the device 110 for X-ray imaging and/or the one or more terminals 130 may be connected to the processing device 140 via the network 120. As another example, the device 110 for X-ray imaging and/or the one or more terminals 130 may be directly connected to the processing device 140.

The device 110 for X-ray imaging may be configured to scan an object using an X-ray and generate image data for generating one or more images related with the object. In some embodiments, the device 110 for X-ray imaging may transmit the image data to the processing device 140 for further processing (e.g., generating one or more images). In some embodiments, the image data related to the object and/or the one or more images may be stored in the storage device 150 and/or the processing device 140.

As shown in FIG. 1, in some embodiments, the device 110 for X-ray imaging may include a C-arm X-ray scanner. In some embodiments, the device 110 for X-ray imaging may include a computed tomography (CT) scanner, a digital radiography (DR) scanner (e.g., a mobile digital radiography scanner), a digital subtraction angiography (DSA) scanner, a dynamic spatial reconstruction (DSR) scanner, an X-ray microscopy scanner, a multimodal scanner, or the like, or a combination thereof. Exemplary multimodal scanners may include a computed tomography-positron emission tomography (CT-PET) scanner, a computed tomography-magnetic resonance imaging (CT-MRI) scanner, or the like.

The device 110 for X-ray imaging may include a support member 111, an X-ray source 112, and a detector 113. The support member 111 may be configured to support the X-ray source 112 and the detector 113. In some embodiments, the support member 111 may be a C shape as shown in FIG. 1. Alternatively, the support member 111 may be a cylindrical shape, an O-shape, a U-shape, a G-shape, etc., or any combination thereof.

In some embodiments, the X-ray source 112 and the detector 113 may be connected to the support member 111. For example, the support member 111 may be a C-shape, a U-shape, a G-shape, etc. The support member 111 may have a first end and a second end. The first end may be connected to the X-ray source 112, and the second end may be connected to the detector 113. As another example, the support member 111 may have an O-shape. The X-ray source 112 and the detector 113 may be attached to the support member 111 and spaced apart from each other. For example, the detector 113 may be disposed opposite the X-ray source 112, and a line connecting the detector 113 and the X-ray source 112 may pass through a center of the O-shape. In some embodiments, the detector 113 and the X-ray source 112 may be separated by a space. The space may be configured to hold one or more objects to be scanned.

In some embodiments, the X-ray source 112 and the detector 113 may be moved with the support member 111. For example, the X-ray source 112 and the detector 113 may be moved with the support member 111 using a movable device (e.g., a vehicle body or wheels) mounted on the device 110 for X-ray imaging. In some embodiments, the X-ray source 112 and/or the detector 113 may be indirectly connected to the support member 111. Merely by way of example, the device 110 for X-ray imaging may include a robotic arm (not shown in FIG. 1). The robotic arm may include an end connected to the support member 111. The robotic arm may also include another end connected to the X-ray source 112. In some embodiments, the robotic arm may be movable and/or retractable.

The X-ray source 112 may emit one or more X-rays to the object. In some embodiments, the X-ray source 112 may include a tube (e.g., a cold cathode ionization tube, a high-vacuum hot cathode tube, a rotating anode tube, or the like). The tube may be powered by a high voltage generator and emit X-rays that may be detected by the detector 113. The X-rays emitted by the X-ray source 112 may be guided to form a beam with a shape (e.g., a linear shape, a narrow pencil shape, a narrow fan shape, a fan shape, a conical shape, a wedge shape, an irregular shape, or the like, or any combination thereof).

The detector 113 may detect radioactive rays emitted from the X-ray source 112. In some embodiments, the detector 113 may be configured to generate an analog electrical signal that indicates received X-rays. The analog electrical signal may include an attenuated beam and an intensity of the X-rays passing through the object. In some embodiments, the detector 113 may include one or more detector units. The detector units may include scintillation detectors (e.g., cesium iodide detectors), gas detectors, or the like. The pixels of the detectors may be represented by a count of minimum detector units. The detector units of the detector 113 may be arranged in a single row, two rows, or other count of rows. An X-ray detector may be one-dimensional, two-dimensional, or three-dimensional.

The network 120 may include any suitable network that may facilitate an exchange of information and/or data of the system 100 for X-ray imaging. In some embodiments, one or more components (e.g., the device 110 for X-ray imaging, the one or more terminals 130, the processing device 140, the storage device 150, or the like) of the system 100 for X-ray imaging may communicate information and/or perform a data interaction with other components of the system 100 for X-ray imaging via the network 120. For example, the processing device 140 may obtain image data (e.g., a grayscale of a current frame) from the device 110 for X-ray imaging via the network 120. As another example, the processing device 140 may obtain user instructions from the one or more terminals 130 via the network 120. In some embodiments, the network 120 may include one or more network access points. For example, the network 120 may include wired and/or wireless network access points, such as a base station and/or an Internet exchange point, through which the one or more components of the system 100 for X-ray imaging may be connected to the network 120 to exchange data and/or information.

The one or more terminals 130 may include a mobile device 131, a tablet computer 132, a laptop computer 133, etc., or any combination thereof. In some embodiments, the mobile device 131 may include a smart home device, a wearable device, a mobile device, a virtual reality device, an augmented reality device, etc., or any combination thereof. In some embodiments, the one or more terminals 130 may be part of the processing device 140.

In some embodiments, the one or more terminals 130 may control an operation of one or more components (e.g., the device 110 for X-ray imaging) of the system 100 for X-ray imaging. For example, a user may set an operating state and/or an operating parameter of the device 110 for X-ray imaging via the one or more terminals 130. For example, the user may set an initial performing parameter of the device 110 for X-ray imaging. In some embodiments, the one or more terminals 130 may be integrated into the device 110 for X-ray imaging. For example, the one or more terminals 130 may be a control panel mounted on the device 110 for X-ray imaging, which may be configured to perform functions of the one or more terminals 130 disclosed in the present disclosure.

The processing device 140 may process data and/or information obtained from the device 110 for X-ray imaging, the one or more terminals 130, and/or the storage device 150. For example, the processing device 140 may process image data generated by the device 110 for X-ray imaging to generate an image. As another example, the processing device 140 may obtain a grayscale of a current frame of an image of the object. In some embodiments, the processing device 140 may be a single server or a group of servers.

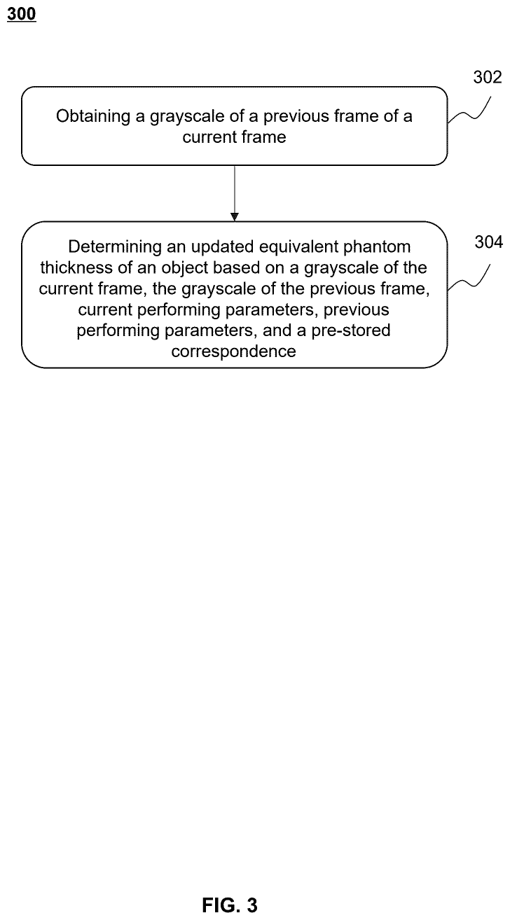

The group of servers may be centralized or distributed. In some embodiments, the processing device 140 may be local or remote. In some embodiments, the processing device 140 may be implemented on a cloud platform. In some embodiments, the processing device 140 may be implemented by a computing device 300 as shown in FIG. 3, which has one or more components.

The storage device 150 may store data, instructions, and/or any other information. In some embodiments, the storage device 150 may store data obtained from the one or more terminals 130 and/or the processing device 140. In some embodiments, the storage device 150 may store data and/or instructions that the processing device 140 may perform or use to perform exemplary processes described in the present disclosure. In some embodiments, the storage device 150 may include a mass memory, a removable memory, a volatile read-write memory, a read-only memory (ROM), etc., or any combination thereof. In some embodiments, the storage device 150 may be implemented on a cloud platform.

In some embodiments, the storage device 150 may be connected to the network 120 to communicate with one or more other components (e.g., the processing device 140, the one or more terminals 130, etc.) of the system 100 for X-ray imaging. The one or more components of the system 100 for X-ray imaging may access data or instructions stored in the storage device 150 via the network 120. In some embodiments, the storage device 150 may be directly connected to or in communication with the one or more other components (e.g., the processing device 140, the one or more terminals 130, etc.) of the system 100 for X-ray imaging. In some embodiments, the storage device 150 may be a part of the processing device 140.

The above descriptions are intended to be illustrative and not to limit the scope of the present disclosure. Many substitutions, modifications, and variations may be apparent to those skilled in the art. The features of the embodiments described herein, structures, processes, and other features may be combined in various ways to obtain additional and/or alternative exemplary embodiments. For example, the processing device 140 and the device 110 for X-ray imaging may be integrated into a single device. These variations and modifications, however, will not be beyond the scope of the present disclosure.

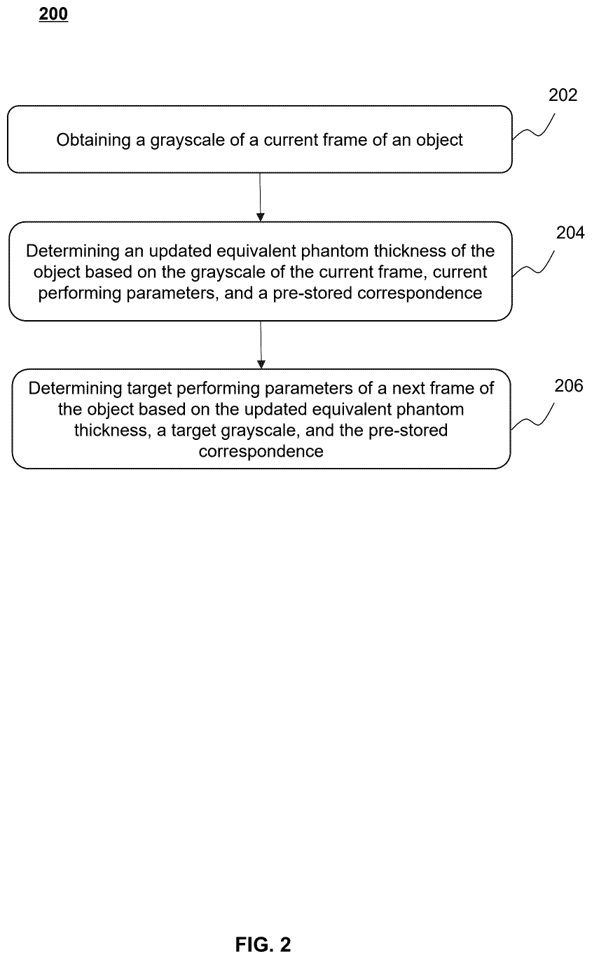

FIG. 2 is a flowchart illustrating an exemplary process for X-ray imaging according to some embodiments of the present disclosure. As shown in FIG. 2, a process 200 may include following operations. In some embodiments, the process 200 may be performed by a processing device (e.g., the processing device 140) or a system for X-ray imaging (e.g., the system 100 for X-ray imaging).

In operation 202, a grayscale of a current frame of an object may be obtained.

The object may include a patient or other medical experimental subjects (e.g., animals such as test mice), etc. The object may also be a portion of the patient or the other medical experimental subjects, including organs and/or tissues (e.g., the heart, lungs, ribs, the abdominal cavity, or the like). The object may also be a medical experimental phantom, e.g., a water phantom, etc.

The current frame may be obtained by performing X-ray imaging on the object based on current performing parameters. For example, the current frame may be an image obtained by an X-ray device through performing X-ray imaging on the object using the current performing parameters.

The current performing parameters may be parameters information loaded by the X-ray device when performing the X-ray imaging. In some embodiments, the performing parameters may include a tube voltage, a tube current, and a performing time of an X-ray tube.

The grayscale may be a brightness level or a gray level of each pixel in an image. In digital images, the grayscale may be usually expressed as an integer value from 0 to 255, where 0 represents black (darkest) and 255 represents white (brightest). The grayscale of an X-ray image may be determined by measuring an amount of radiations received by an X-ray sensor. In a process for X-ray imaging, an X-ray beam passing through an object may be received by a detector, which may convert the received X-ray beam into an electrical signal and determine the grayscale of each pixel based on an intensity of the electrical signal.

In some embodiments, the X-ray device may be capable of scanning the object based on initial performing parameters to obtain the grayscale of the current frame. The initial performing parameters may include a tube voltage, a tube current, and a performing time of the X-ray tube in an initial state.

In some embodiments, the initial performing parameters may be set based on empirical values or may be obtained based on a planning protocol of the object. In some embodiments, the planning protocol may also include an initial phantom thickness and a target grayscale. More details regarding the initial phantom thickness and the target grayscale may be found elsewhere in the present disclosure (e.g., the description in connection with operation 206).

In some embodiments, the initial performing parameters may also be obtained based on previous performing parameters and a grayscale of a previous frame. For example, the processing device may judge whether the performing parameters need to be updated based on the grayscale of the previous frame and the target grayscale, and when updating is required, the previous performing parameters of the previous frame may be updated, and the updated previous performing parameters may be designated as the initial performing parameters. More details regarding the previous frame may be found elsewhere in the present disclosure (e.g., the description in connection with FIG. 3).

In operation 204, an updated equivalent phantom thickness of the object may be determined based on the grayscale of the current frame, the current performing parameters, and a pre-stored correspondence.

The phantom thickness refers to a thickness of an object to be scanned in a direction of an X-ray beam. When an X-ray scan is performed, the X-ray beam may pass through the object to be scanned and interact with tissues or structures inside the object. The phantom thickness may determine a distance that the X-ray beam may travel inside the object.

An equivalent phantom refers to a substance with a specific density and thickness that may be configured to simulate absorption and scattering behaviors of human tissues in the X-ray imaging. The equivalent phantom thickness refers to a thickness of the equivalent phantom in the direction of the X-ray beam.

The pre-stored correspondence refers to an interrelationship or a mapping relationship between the equivalent phantom thickness and performing parameters of X-rays. The pre-stored correspondence may indicate a correlation between the equivalent phantom thickness and the performing parameters of the X-rays.

In some embodiments, the pre-stored correspondence may reflect a correlation between dose values corresponding to a plurality of equivalent phantom thicknesses and a plurality of performing parameters. For example, the pre-stored correspondence may include dose values under the plurality of equivalent phantom thicknesses and a plurality of tube voltages, a plurality of tube currents, and a plurality of performing times. The values may be specific, and different values have a one-to-one pre-stored correspondence with the plurality of equivalent phantom thicknesses and different tube voltages, tube currents, and performing times.

It should be noted that the plurality of tube voltages, the plurality of tube currents, and the plurality of performing times satisfy a preset relationship, which may be determined by an ABS (Automatic Brightness Stabilization) brightness adjustment curve. The ABS brightness adjustment curve may be used to limit a degree of freedom that may be loaded into parameters of the X-ray tube and satisfy a grayscale requirement for reading a film. Therefore, the ABS brightness adjustment curve may limit the relationship between the plurality of tube voltages, the plurality of tube currents, and the plurality of performing times. In the present disclosure, performing parameters may be represented by a corresponding tube voltage, and a tube current. A performing time corresponding to the tube voltage may be constrained by the ABS brightness adjustment curve.

The dose values refer to actual values of X-ray radiation doses measured at a specific location or a received object with a same phantom thickness when the X-ray device loads a plurality of performing parameters.

In some embodiments, the processing device may determine the updated equivalent phantom thickness of the object based on the grayscale of the current frame, the current performing parameters, and the dose values under the plurality of equivalent phantom thicknesses and the plurality of performing parameters. For example, the pre-stored correspondence may be as shown in Table 1, where a horizontal direction of Table 1 includes a plurality of phantom thicknesses, a vertical direction of Table 1 includes tube voltages in a plurality of current performing parameters, and cells in Table 1 (only a few examples are shown here, others are represented by spaces) are dose values. For example, a dose value at a phantom thickness of 5 cm and a tube voltage of 70 kv may be A, a dose value at a phantom thickness of 5 cm and a tube voltage of 90 kv may be B, a dose value at a phantom thickness of 10 cm and a tube voltage of 70 kv may be C, and a dose value at a phantom thickness of 10 cm and a tube voltage of 90 kv may be D, etc.

TABLE 1
Value data of doses under different phantom
thicknesses and tube voltages (kv)

phantom thickness

| | | | | | | | | |
|---|---|---|---|---|---|---|---|---|
| | 5 | 10 | 15 | 20 | 25 | 30 | 35 | 40 |
| kv | cm | cm | cm | cm | cm | cm | cm | cm |

| | | |
|---|---|---|
| 40 | | |
| 45 | | |
| 50 | | |
| 55 | | |
| 60 | | |
| 65 | | |
| 70 | A | C |
| 75 | | |
| 80 | | |
| 85 | | |
| 90 | B | D |
| 95 | | |
| 100 | | |
| 105 | | |
| 110 | | |
| 115 | | |
| 120 | | |
| 125 | | |

The processing device may determine the updated equivalent phantom thickness of the object based on the grayscale of the current frame, the current performing parameters, and the dose values under the plurality of equivalent phantom thicknesses and the plurality of performing parameters by querying a preset correspondence (e.g., Table 1). For example, when the tube voltage (kv) is certain, performing parameters and dose values corresponding to different phantom thicknesses may be viewed along the horizontal direction. According to the current performing parameters and the grayscale of the current frame, a dose value may be calculated, and a phantom thickness corresponding to the dose value in the table may be found as the updated equivalent phantom thickness.

In some embodiments, the pre-stored correspondence may include normalized values of dose attenuations under the plurality of equivalent phantom thicknesses and the plurality of performing parameters.

The normalized values of the dose attenuations refer to values obtained by linearly transforming the dose values in a certain way. For example, it may be assumed that a dose value under a tube voltage of 70 kv and an equivalent phantom thickness of 5 cm is A, and a normalized value corresponding to A is 1 (e.g., taking the value of A as a reference). When a dose value under a tube voltage of 70 kv and an equivalent phantom thickness of 10 cm is B, a normalized value of a dose attenuation under the tube voltage of 70 kv and the equivalent phantom thickness of 10 cm may be obtained through dividing B by A. For example, the normalized value may be 0.8. At this time, the normalized values of the dose attenuations under the plurality of equivalent phantom thicknesses and the plurality of performing parameters are shown in Table 2.

TABLE 2
Normalized data of dose attenuations under different
phantom thicknesses and tube voltages (kv)

phantom thickness

| | | | | | | | | |
|---|---|---|---|---|---|---|---|---|
| | 5 | 10 | 15 | 20 | 25 | 30 | 35 | 40 |
| kv | cm | cm | cm | cm | cm | cm | cm | cm |

| | | |
|---|---|---|
| 40 | | |
| 45 | | |
| 50 | | |
| 55 | | |
| 60 | | |
| 65 | | |
| 70 | 1 | 0.8 |
| 75 | | |
| 80 | | |
| 85 | | |
| 90 | 1.5 | 1.1 |
| 95 | | |
| 100 | | |
| 105 | | |
| 110 | | |
| 115 | | |
| 120 | | |
| 125 | | |

In some embodiments, the processing device may determine corresponding normalized values of dose attenuations based on the grayscale of the current frame, the grayscale of the previous frame, the current performing parameters, the previous performing parameters of the previous frame, and the dose values under the plurality of equivalent phantom thicknesses and the plurality of performing parameters, and determine the updated equivalent phantom thickness of the object by querying a preset correspondence (e.g., Table 2).

For example, it may be supposed that the previous performing parameters of the previous frame is 70 kv, the equivalent phantom thickness is 5 cm, the grayscale of the previous frame is 0.5, current performing parameters of the current frame is 90 kv, and the grayscale of the current frame is 0.75. At this time, a ratio of the grayscale of the current frame to that of the previous frame is 1.5. The ratio may reflect that when the tube voltage in the performing parameters is adjusted from 70kv to 90kv, the grayscale is changed by a multiple of 1.5. According to the multiple, it may be seen from Table 2 that when the phantom thickness is 5 cm, the multiple of the normalized value of the dose attenuation when the tube voltage is adjusted from 70 kv to 90 kv is also 1.5. At this time, the corresponding phantom thickness (i.e., 5 cm) in Table 2 may be designated as the updated equivalent phantom thickness.

In operation 206, target performing parameters of a next frame of the object may be determined based on the updated equivalent phantom thickness, a target grayscale, and the pre-stored correspondence.

The target grayscale refers to a desired grayscale of an image obtained by X-ray imaging.

In some embodiments, after determining the updated equivalent phantom thickness, a tube voltage corresponding to the target grayscale may be found according to the pre-stored correspondence. The tube current and the performing time corresponding to the tube voltage may be determined based on the ABS brightness adjustment curve (hereinafter referred to as an ABS curve). For example, if there are a plurality of working points in the ABS curve that meet a brightness requirement, and the plurality of working points correspond to a plurality of working performing parameters, then the working parameters corresponding to the working points may be selected according to different image viewing requirements of a user. Merely by way of example, if there are two working points of “70 kV 200 mA” and “80 kV 120 mA” on the ABS curve that achieve a same brightness, the two working points may be selected according to requirements of a user. The requirements of the user may include an image viewing requirement (e.g., a requirement of an image quality, which may be expressed by a contrast or a signal-to-noise ratio), a device performance loss requirement (e.g., a requirement of a power), etc.

In some embodiments, the determining the target performing parameters of the next frame of the object based on the updated equivalent phantom thickness, the target grayscale, and the pre-stored correspondence may include: determining whether the grayscale of the current frame is within a target grayscale value interval; and in response to determining that the grayscale of the current frame is within the target grayscale value interval, designating the current performing parameters as the target performing parameters of the next frame.

The target grayscale value interval refers to a range of expected grayscale values of performing parameters. In some embodiments, after obtaining the grayscale of the current frame, since the grayscale value is relatively absolute, when sending the grayscale value to a high-voltage generator of an imaging device, a target grayscale value and a control accuracy also need to be input. The control accuracy is a floating space of the target grayscale value. In some embodiments, the grayscale value may be normalized and sent to the high-voltage generator. If the target grayscale value is 1000 and the grayscale of the current frame is 200, the normalized value is 0.2; then the high-voltage generator may compare the normalized value (i.e., 0.2) with a preset normalized target grayscale value interval (e.g., 0.9-1.1). In this embodiment, there may be no need to input the control accuracy to the high-voltage generator.

In some embodiments, the target grayscale value interval may be determined according to different scanning protocols.

In some embodiments, the processing device may directly compare the grayscale of the current frame to the target grayscale value interval to determine whether the grayscale of the current frame is within the target grayscale value interval. When the grayscale of the current frame is within the target grayscale value interval, it may be assumed that the current performing parameters of the current frame may satisfy a preset expectation. That is, the current performing parameters may continue to be used and designated as the target performing parameters of the next frame.

In some embodiments, the processing device may, in response to determining that the grayscale of the current frame is out of the target grayscale value interval, determine whether the grayscale of the current frame is within a closed-loop control grayscale range.

The closed-loop control grayscale range refers to a grayscale interval used to determine whether to enter a closed-loop control. The closed-loop control grayscale range has a minimum value and a maximum value. The grayscale of the current frame being within the closed-loop control grayscale range means that the grayscale of the current frame is greater than or equal to the minimum value and is less than or equal to the maximum value, that is, the grayscale of the current frame is within a grayscale interval of the closed-loop control.

In some embodiments, in response to determining that the grayscale of the current frame is not within the closed-loop control grayscale range, the processing device may determine performing parameters that satisfy the target grayscale as the target performing parameters based on the grayscale of the current frame, the current performing parameters, the updated equivalent phantom thickness, and the pre-stored correspondence. For example, when determining that the grayscale of the current frame is not within the closed-loop control grayscale range, i.e., when the grayscale of the current frame is less than the minimum value or greater than the maximum value, an operation of updating the equivalent phantom may be performed. Then, based on the grayscale of the current frame and the current performing parameters, the equivalent phantom thickness may be updated according to the pre-stored correspondence, and based on the updated equivalent phantom thickness, the performing parameters that satisfy the target grayscale may be determined.

In some embodiments, in response to determining that the grayscale of the current frame is within the closed-loop control grayscale range, the processing device may determine the target performing parameters of the next frame through closed-loop control based on the grayscale of the current frame, the target grayscale, and the current performing parameters. For example, the target performing parameters of the next frame may be determined by closed-loop control. The closed-loop control has an advantage of a good regulation stability. In this embodiment, a change stability of the image brightness can be improved by adding an operation of determining the grayscale using the closed-loop control and updating the performing parameters based on the closed-loop control in response to determining that the grayscale of the current frame is within the closed-loop control grayscale range.

In some embodiments, the closed-loop control may be performed by updating the performing parameters based on the grayscale of the current frame, the target grayscale, and the current performing parameters after a closed-loop computation, and the closed-loop process may be performed in a manner of Proportional-Integral-Derivative (PID) or successive approximation. Then the next frame may be obtained by performing X-ray imaging according to the updated performing parameters, and a subsequent control strategy may be determined based on the grayscale of the next frame.

In some embodiments of the present disclosure, an attenuation law of different objects to X-rays may be obtained by an offline manner, and the correspondence reflecting the dose values or the normalized values of the dose attenuations under the plurality of equivalent phantom thicknesses and the plurality of performing parameters may be determined. The equivalent phantom thickness of the object may be updated according to the online pre-stored correspondence, and then the performing parameters of the X-rays may be updated according to the equivalent phantom thickness and a distance from the target grayscale, which may achieve a purpose of taking into account the rapidity and stability of ABS adjustment.

FIG. 3 is a flowchart illustrating an exemplary process for determining an updated equivalent phantom thickness according to some embodiments of the present disclosure.

In operation 302, a grayscale of a previous frame of a current frame may be obtained.

In some embodiments, the previous frame may be an image obtained by performing X-ray imaging on the object based on previous performing parameters of the previous frame. The previous frame refers to an image reconstructed by image data acquired at a time point before a current time point when acquiring image data of the current frame. In some embodiments, the processing device may obtain the previous frame by accessing the storage device, calling a data interface, etc., and calculating to obtain the grayscale of the previous frame based on the previous frame.

In some embodiments, the grayscale of the previous frame may also be obtained and stored when the X-ray device performs imaging on the object, and the processing device may read directly to obtain the grayscale of the previous frame.

In operation 304, the updated equivalent phantom thickness of the object may be determined based on the grayscale of the current frame, the grayscale of the previous frame, the current performing parameters, the previous performing parameters, and the pre-stored correspondence.

In some embodiments, the processing device may determine a ratio of the grayscale of the current frame to the grayscale of the previous frame based on the grayscale of the current frame, the grayscale of the previous frame, the current performing parameters, and the previous performing parameters, and determine the updated equivalent phantom thickness of the object based on the ratio.

In some embodiments, the processing device may determine the ratio of the grayscale of the current frame to the grayscale of the previous frame. For example, the processing device may divide the grayscale of the current frame by the grayscale of the previous frame to obtain the ratio. The processing device may determine the updated equivalent phantom thickness of the object based on the ratio of the grayscale of the current frame to the grayscale of the previous frame, the current performing parameters, the previous performing parameters, and the pre-stored correspondence.

In some embodiments, the ratio may also be referred to as a first modulation strategy.

In some embodiments, when the current performing parameters of the current frame remains unchanged, the processing device may determine an equivalent phantom thickness corresponding to the ratio of the grayscale of the current frame to the grayscale of the previous frame according to the pre-stored correspondence and designate the equivalent phantom thickness as the updated equivalent phantom thickness.

The specific process for determining the updated equivalent phantom thickness may be found in descriptions related to operation 204, which is not repeated here.

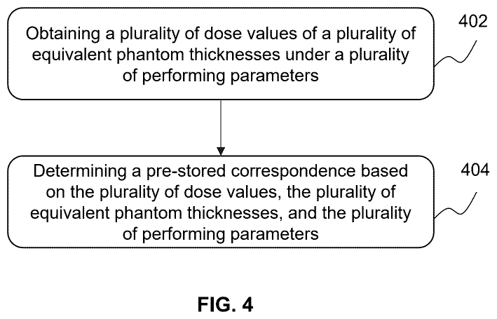

FIG. 4 is a flowchart illustrating an exemplary process for determining a correspondence according to some embodiments of the present disclosure.

In operation 402, a plurality of dose values of a plurality of equivalent phantom thicknesses under a plurality of performing parameters may be obtained.

In some embodiments, a plurality of equivalent phantoms with a plurality of thicknesses may be pre-constructed phantoms, e.g., water phantoms, etc. The dose values may be obtained by performing X-ray imaging on the equivalent phantoms with a plurality of thicknesses under the plurality of performing parameters.

For example, the processing device may adjust the performing parameters of the X-rays by sequentially obtaining the equivalent phantoms with a plurality of phantom thicknesses. For example, when the phantom thickness remains unchanged at 5 cm, a plurality of times of X-ray imaging may be performed in a voltage range of 40 kv-125 kv according to a tube voltage interval of 5 kv to obtain a plurality of dose values of equivalent phantoms with a same thickness under a plurality of performing parameters. A plurality of times of X-ray imaging may be performed in the same way on equivalent phantoms with a plurality of thicknesses to obtain a plurality of dose values of the equivalent phantoms with the plurality of thicknesses under the plurality of performing parameters.

There is a certain correspondence between the grayscale value and the dose value, and according to the correspondence, a dose value may be determined based on a grayscale value corresponding to the dose value. In the embodiments of the present disclosure, the dose value under each performing parameter is represented by a corresponding grayscale value. That is, the grayscale value and the dose value can be converted to each other without causing confusion.

It should be noted that the plurality of thicknesses and the plurality of performing parameters in the above examples are given for exemplary purposes only. For example, a phantom thickness interval may be 10 cm and the tube voltage interval may be 10 kv, which is not limited in the present disclosure.

In operation 404, the pre-stored correspondence may be determined based on the plurality of dose values, the plurality of equivalent phantom thicknesses, and the plurality of performing parameters.

In some embodiments, the processing device may construct a table by using the equivalent phantom thicknesses and the performing parameters as table headers, respectively, and fill dose values of the equivalent phantoms with the same thickness under the plurality of performing parameters into the table to obtain the pre-stored correspondence.

In some embodiments, the representation of the pre-stored correspondence may also be in the form of a curves, a graph, or the like, which is not limited in the present disclosure.

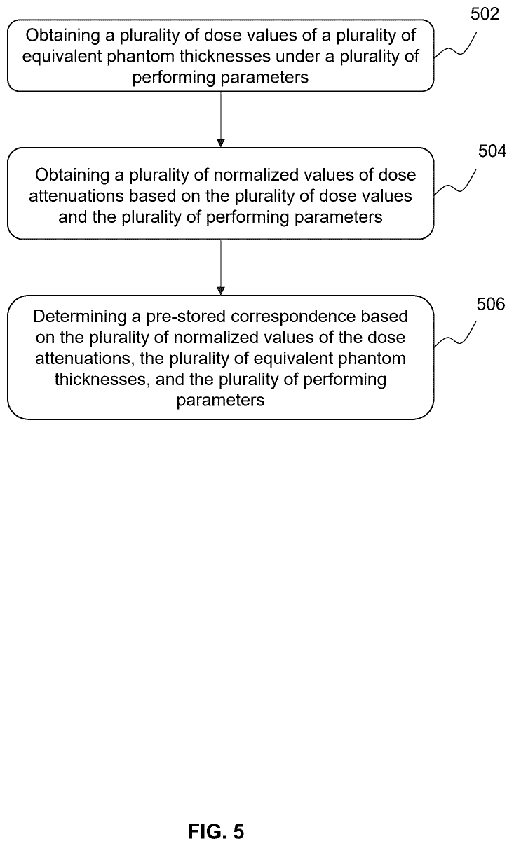

FIG. 5 is a flowchart illustrating another exemplary process for determining a correspondence according to some embodiments of the present disclosure.

In operation 502, a plurality of dose values of a plurality of equivalent phantom thicknesses under a plurality of performing parameters may be obtained.

In some embodiments, the dose values may be obtained by performing X-ray imaging on a plurality of equivalent phantoms with a plurality of thicknesses under the plurality of performing parameters.

More details regarding operation 502 may be found elsewhere in the present disclosure (e.g., the description in connection with operation 402).

In operation 504, a plurality of normalized values of dose attenuations may be obtained based on the plurality of dose values and the plurality of performing parameters.

In some embodiments, a certain performing parameter (e.g., 80 kv) may be selected as a benchmark, and dose attenuation values corresponding to other performing parameters may be divided by a dose attenuation value corresponding to 80 kv under the same equivalent phantom thickness to obtain the normalized values of the dose attenuations in the pre-stored correspondence. By performing a normalization process, it can be easier to determine the updated equivalent phantom thickness based on the pre-stored correspondence according to grayscales of previous and current frames and the performing parameters.

For example, the processing device may determine the plurality of normalized value of the dose attenuations by linearly normalizing the plurality of dose values based on the plurality of performing parameters. Since there is a certain linear relationship between different equivalent phantom thicknesses, tube voltages, tube currents, performing times, and dose values, the normalization here is a normalization of the linear relationship.

In some embodiments, a linear normalization result may be directly used as the normalized value of the dose attenuation.

In some embodiments, the processing device may determine the plurality of normalized values of the dose attenuations by non-linearly normalizing the plurality of dose values based on reference performing parameters.

The reference performing parameters may be performing parameters selected as reference data when non-linearly normalizing. In some embodiments, the reference performing parameters may be any specified performing parameters other than the above plurality of performing parameters.

In some embodiments, the processing device may perform a linear normalization based on the reference performing parameters to obtain a linear normalization result of the reference performing parameters.

The non-linear normalization refers to a normalization between the linear normalization results of the performing parameters and the linear normalization result of the reference performing parameters. Since there is no linear relationship between the reference performing parameters and the performing parameters, by taking the linear normalization result of the reference performing parameters as a reference, the plurality of normalization results of the plurality of performing parameters may be divided by the linear normalization result of the reference performing parameters to obtain the plurality of normalized values of the dose attenuations.

In operation 506, the pre-stored correspondence may be determined based on the plurality of normalized values of the dose attenuations, the plurality of equivalent phantom thicknesses, and the plurality of performing parameters.

In some embodiments, the processing device may construct a table by using the equivalent phantom thicknesses and the performing parameters as table headers, respectively, and fill the plurality of normalized values of the dose attenuations into the table to obtain the pre-stored correspondence.

It should be noted that the foregoing descriptions of the respective processes are for the purpose of exemplification and illustration only and do not limit the scope of application of the present disclosure. For those skilled in the art, various corrections and changes may be made to the process of FIG. 5 under the guidance of the present disclosure. However, these corrections and changes remain within the scope of the present disclosure. For example, storage operations may be added between the above operations, etc.

Based on the same inventive concept, the following embodiments are also disclosed in the present disclosure.

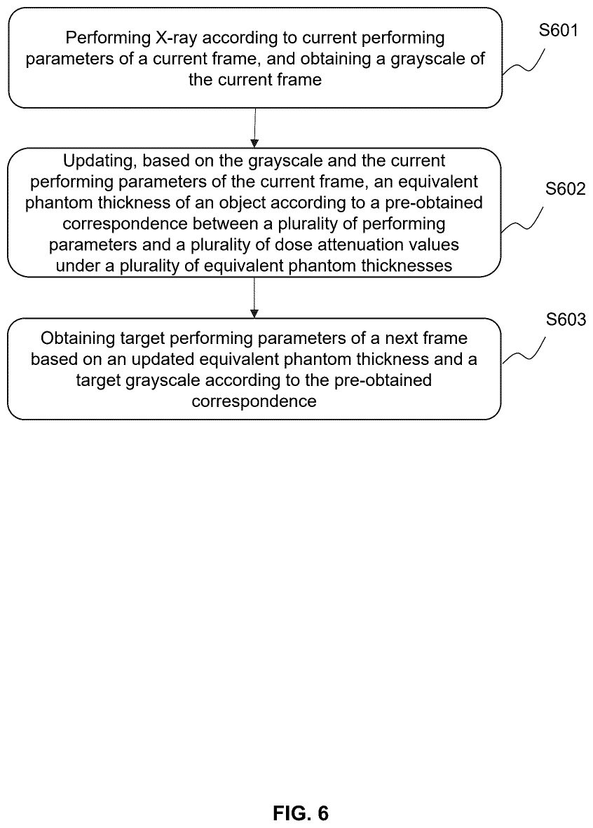

FIG. 6 is a flowchart illustrating an exemplary control process for a system for X-ray imaging according to some embodiments of the present disclosure. As shown in FIG. 6, the process may include the following operations:

In operation S601, X-ray imaging may be performed according to current performing parameters of a current frame, and a grayscale of the current frame may be obtained. Optionally, the current performing parameters may include a tube voltage, a tube current, and a performing time of an X-ray tube.

In some embodiments, the current performing parameters may be obtained based on a currently selected loading protocol, which is optionally set based on empirical values.

In some embodiments, the current performing parameters may be obtained based on previous performing parameters and a grayscale of a previous frame. Specifically, the process may further include an operation of obtaining the current performing parameters of the current frame before operation S601.

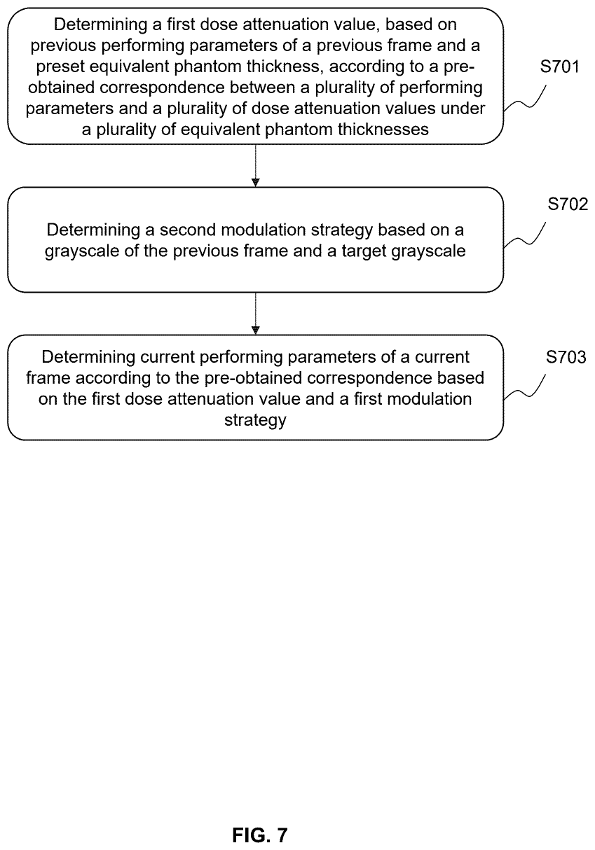

FIG. 7 is a flowchart illustrating an exemplary process for obtaining current performing parameters of a current frame according to some embodiments of the present disclosure. As shown in FIG. 7, the process for obtaining current performing parameters of a current frame may specifically include following operations.

In operation S701: a first dose attenuation value may be determined, based on previous performing parameters of a previous frame and a preset equivalent phantom thickness, according to a pre-obtained correspondence between a plurality of performing parameters and a plurality of dose attenuation values under a plurality of equivalent phantom thicknesses.

The correspondence adopted in operation S701 may include two dimensions (i.e., equivalent phantom thicknesses and performing parameters), in which a plurality of dose attenuation values of the current frame under the plurality of equivalent phantom thicknesses and the plurality of performing parameters may be recorded.

An equivalent phantom may be configured to simulate an object, and a plurality of objects may be simulated using equivalent phantoms with a plurality of thicknesses. For example, the equivalent phantoms with a plurality of thicknesses may be configured to simulate different parts of a body, or objects to be detected with different body sizes. The dose attenuation value may be positively correlated with the grayscale of the frame, and a change in the dose attenuation value may directly reflect a change in the grayscale of the frame. In the correspondence, the performing parameters may be specifically the tube voltage of the X-ray tube, i.e., the correspondence may record a plurality of dose attenuation values under a plurality of tube voltages and a plurality of equivalent phantom thicknesses.

Optionally, the correspondence may be pre-stored in the form of a table. For ease of exposition, Table 3 is used as an example.

TABLE 3
Correspondence table among a plurality of dose attenuation
values, a plurality of equivalent phantom thicknesses,
and a plurality of tube voltages

Equivalent phantom Thickness

| Tube Voltage | 15 cm | 20 cm | 25 cm |
|---|---|---|---|
| 40 kv | A1 | A2 | A3 |
| 45 kv | B1 | B2 | B3 |
| 50 kv | C1 | C2 | C3 |
| 55 kv | D1 | D2 | D3 |

In conjunction with Table 3, in this correspondence table, a unique dose attenuation value may be located based on different tube voltages and equivalent phantom thicknesses with the tube voltages as a first dimension and the equivalent phantom thicknesses as a second dimension. In operation S701, the first dose attenuation value may be determined from the correspondence table based on the previous performing parameters of the previous frame (i.e., the tube voltage loaded in the previous frame) and the preset phantom thickness.

It should be noted that Table 3 is only an exemplary demonstration. A step value and a value range of the equivalent phantom thickness, and a step value and a value range of the tube voltage in the correspondence are not specifically limited, which may be classified according to the actual needs.

In addition, it is stated that the tube voltage, the tube current, and the performing time satisfy a preset relationship, which may be specified by an ABS brightness adjustment curve. The ABS brightness adjustment curve may be configured to limit a degree of freedom that may be loaded into parameters of the X-ray tube and satisfy a grayscale requirement for viewing the image. Then working point parameters for actual imaging may be determined based on the ABS brightness adjustment curve. The working point parameters may include a tube voltage, a tube current, and a performing time.

For example, if there are a plurality of working points in the ABS curve that meet a brightness requirement, and the plurality of working points correspond to a plurality of working performing parameters, then the working parameters corresponding to the working points may be selected according to different image viewing requirements of a user. Merely by way of example, if there are two working points of “70 kV 200 mA” and “80 kV 120 mA” on the ABS curve that achieve a same brightness, the two working points may be selected according to requirements of a user. The requirements of the user may include an image viewing requirement (e.g., a requirement of an image quality, which may be expressed by a contrast or a signal-to-noise ratio), a device performance loss requirement (e.g., a requirement of a power), etc.

In operation S702: a second modulation strategy may be determined based on the grayscale of the previous frame and a target grayscale.

The target grayscale refers to a desired grayscale of an image obtained by the X-ray imaging. Optionally, a ratio of the grayscale of the previous frame and the target grayscale may be adopted as the second modulation strategy in operation S702.

In operation S703, the current performing parameters of the current frame may be determined according to the correspondence based on the first dose attenuation value and the first modulation strategy.

In operation S703, the current equivalent phantom thickness is unchanged, and a target dose attenuation value may be determined according to the second modulation strategy and the first dose attenuation value. Specifically, the target dose attenuation value may be determined according to a product of the first dose attenuation value and the second modulation strategy. Furthermore, the tube voltage may be determined in the correspondence table based on the equivalent phantom thickness and the target dose attenuation value. The tube voltage, a tube current, and a performing time adapted to the tube voltage may be configured as the current frame performing parameters.

Referring back to FIG. 6, operation S602 is performed after operation S601 as follows.

In operation S602: based on the grayscale and the current performing parameters of the current frame, the equivalent phantom thickness of the object may be updated according to the pre-obtained correspondence between the plurality of performing parameters and the plurality of dose attenuation values under the plurality of equivalent phantom thicknesses.

The grayscale may be positively related to the dose attenuation value, the tube current, and the performing time. Therefore, according to the pre-obtained correspondence, the equivalent phantom thickness of the object may be updated based on the tube voltage in the current performing parameters of the current frame and the current dose attenuation value.

In some embodiments, the thickness of the equivalent phantom may be preset when imaging the previous frame, at which time the equivalent phantom thickness may be updated in conjunction with a loading situation of the previous frame and a loading situation of the current frame through the operation S602.

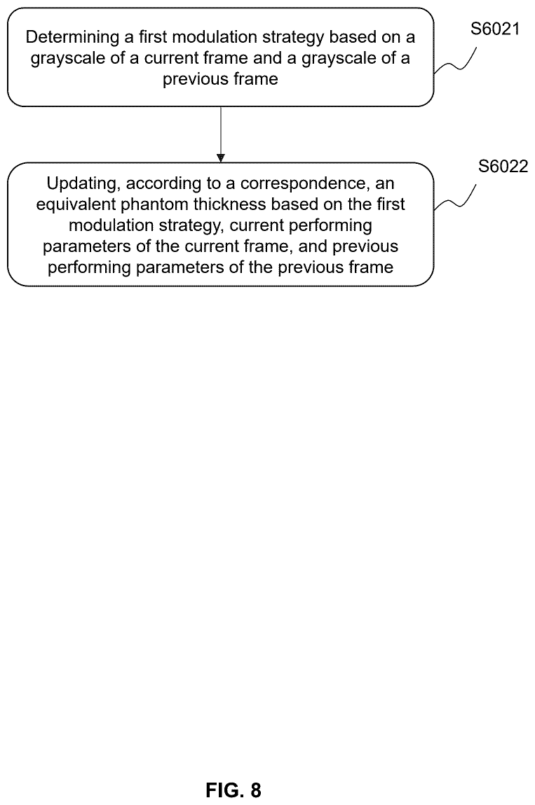

FIG. 8 is a flowchart illustrating operation S602 according to some embodiments of the present disclosure. As shown in FIG. 8, operation S602 may include following contents.

In operation S6021, the first modulation strategy may be determined based on the grayscale of the current frame and the grayscale of the previous frame.

Optionally, in operation S6021, a ratio of the grayscale of the current frame to the grayscale of the previous frame may be determined as the first modulation strategy.

In operation S6022, according to the correspondence, the equivalent phantom thickness may be updated based on the first modulation strategy, the current performing parameters of the current frame, and the previous performing parameters of the previous frame.

Optionally, in operation S6022, according to the correspondence, actual dose attenuation data of the previous frame may be determined based on the previous performing parameters of the previous frame and a preset phantom thickness. According to the correspondence, when the current performing parameters of the current frame is unchanged, an equivalent phantom thickness corresponding to a ratio closest to the first modulation strategy may be determined and designated as the updated equivalent phantom thickness. The ratio is a ratio of dose attenuation data corresponding to the equivalent phantom thickness to the actual dose attenuation data of the previous frame.

In some embodiments, Table 4 is a correspondence table illustrated according to an exemplary embodiment as follows.

TABLE 4
Correspondence table between a plurality of equivalent phantom
thicknesses and a plurality of tube of dose attenuation values

Equivalent phantom Thickness

| Tube Voltage | 20 cm | 25 cm |
|---|---|---|
| 80 kv | 1 | 1 |
| 90 kv | 1.2 | 2 |

Combined with Table 4, the previous performing parameter of the previous frame is 80 kv, the preset equivalent phantom thickness is 25 cm, the grayscale of the previous frame is 0.5, the current performing parameter of the current frame is 90 kv, and the grayscale of the current frame is 0.6. At this time, the first regulation strategy is a ratio of the grayscale of the previous frame to the grayscale of the current frame, i.e., 1.2. Keeping the current performing parameter of the current frame as 90 kv, the equivalent phantom thickness corresponding to the ratio closest to the first modulation strategy may be 20 cm, where the ratio is the ratio of the dose attenuation data corresponding to the equivalent phantom thickness to the actual dose attenuation data of the previous frame.

Referring back to FIG. 6, operation S603 may be performed after operation S602 as follows.

In operation S603, target performing parameters of a next frame may be obtained based on the updated equivalent phantom thickness and the target grayscale according to the correspondence.

Optionally, according to the correspondence table, performing parameters corresponding to dose attenuation data closest to the target grayscale may be determined based on the updated equivalent phantom thickness and configured as the target performing parameters of the next frame.

In summary, the control process for the system for X-ray imaging provided by the embodiments of the present disclosure may update the phantom thickness based on the pre-obtained correspondence to rapidly debug and adapt to the performing parameters of the current object to realize the adjustment of image brightness. In this way, due to the adoption of the pre-obtained correspondence, a speed of adjusting the performing parameters is improved, a stabilization time in a process for automatic brightness adjustment is shortened, and defects in the related technology are overcome to optimize user experience.

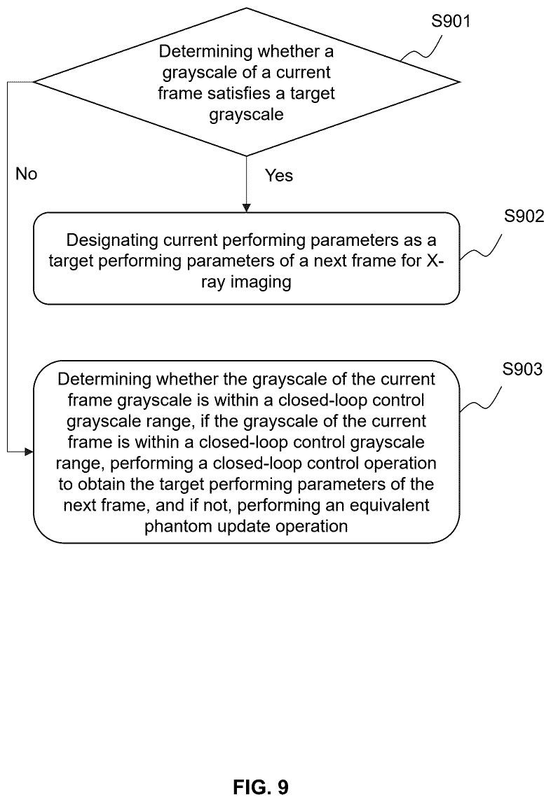

FIG. 9 is a flowchart illustrating another exemplary control process for a system for X-ray imaging according to some embodiments of the present disclosure. As shown in FIG. 9, the process may further include following operations.

In operation S901, whether a grayscale of a current frame satisfies a target grayscale may be determined. If the grayscale of the current frame satisfies the target grayscale, operation S902 may be performed; and if the grayscale of the current frame does not satisfy the target grayscale, operation S903 may be performed.

The target grayscale may be an interval of grayscale values, including a target grayscale minimum and a target grayscale maximum. In operation S901, if the grayscale of the current frame is greater than or equal to the target grayscale minimum and less than or equal to the target grayscale maximum, the target grayscale is satisfied; and if the grayscale of the current frame is less than the target grayscale minimum or greater than the target grayscale maximum, the target grayscale is not satisfied.

In operation S902, the current performing parameters may be configured as the target performing parameters of the next frame for X-ray imaging. At this time, since the grayscale of the current frame satisfies the target grayscale and meets a current grayscale requirement, the X-ray imaging may continue to be performed with the current performing parameters to load the next frame.

In operation S903, whether the grayscale of the current frame grayscale is within a closed-loop control grayscale range may be determined, if the grayscale of the current frame is within a closed-loop control grayscale range, a closed-loop control operation may be performed to obtain the target performing parameters of the next frame, and if not, an equivalent phantom update operation may be performed.

Optionally, the closed-loop control grayscale range has a minimum value and a maximum value, and the grayscale of the current frame being within the closed-loop control grayscale range refers that the grayscale of the current frame is greater than or equal to the minimum value and less than or equal to the maximum value. At this time, the target performing parameters of the next frame may be updated by means of the closed- loop control. The closed-loop control has an advantage of a good regulation stability. In embodiments of the present disclosure, the operation of determining the grayscale based on the closed-loop control may be added, and if the grayscale of the current frame grayscale is within the closed-loop control grayscale range, the performing parameters may be updated based on the closed-loop control, which may improve the change stability of image brightness.

When the grayscale of the current frame is not within the closed-loop control grayscale range (e.g., when the grayscale of the current frame is less than the minimum value or greater than the maximum value), the equivalent phantom update operation may be performed. Specifically, based on the grayscale of the current frame and the current performing parameters of the current frame, the equivalent phantom thickness may be updated by the correspondence. Further, the target performing parameters of the next frame may be obtained based on the updated equivalent phantom thickness and the target grayscale by the correspondence for loading the next frame.

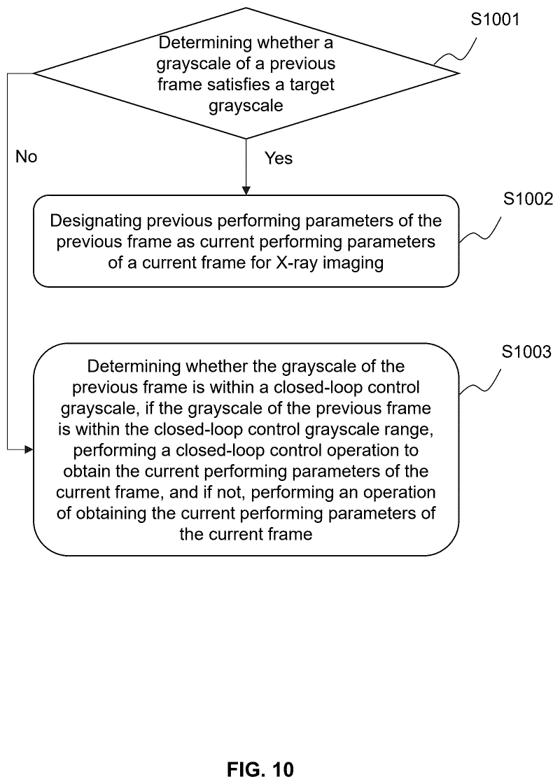

FIG. 10 is a flowchart illustrating another exemplary control process for a system for X-ray imaging according to some embodiments of the present disclosure. As shown in FIG. 10, the process may further include following operations.

In operation S1001, whether a grayscale of a previous frame satisfies a target grayscale may be determined. If the grayscale of the previous frame satisfies the target grayscale, operation S1002 may be performed; and if not, operation S1003 may be performed.

In operation S1002, previous performing parameters of the previous frame may be used as current performing parameters of a current frame for X-ray imaging. At this time, since the grayscale of the current frame satisfies a target grayscale and meets a current grayscale requirement, the X-ray imaging may continue to be performed with the previous performing parameters.

In operation S1003, whether the grayscale of the previous frame is within a closed-loop control grayscale range may be determined, if the grayscale of the previous frame is within the closed-loop control grayscale range, a closed-loop control operation may be performed to obtain the current performing parameters of the current frame, and if not, an operation of obtaining the current performing parameters of the current frame may be performed.

Similarly, if the grayscale of the previous frame is within the closed-loop control grayscale range, the closed-loop control may be configured to obtain the target performing parameters of the next frame to improve adjustment stability of image brightness. If the current performing parameters of the current frame is not within the closed-loop control grayscale range, operations S701-S703 described above may be performed to obtain the current performing parameters to shorten the ABS stabilization time.

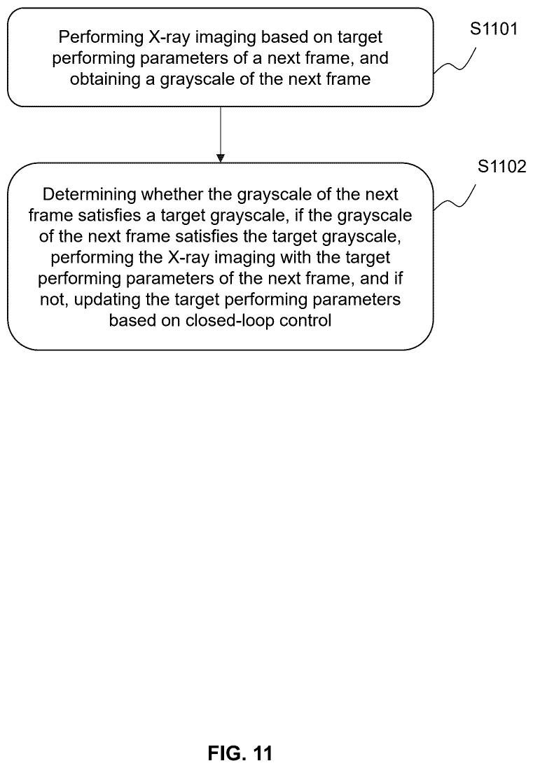

FIG. 11 is a flowchart illustrating another exemplary control process for a system for X-ray imaging according to some embodiments of the present disclosure. As shown in FIG. 11, the process described after operation S603 may further include following operations:

In operation S1101, X-ray imaging may be performed based on target performing parameters of a next frame, and a grayscale of the next frame may be obtained.

In operation S1102, whether the grayscale of the next frame satisfies a target grayscale may be determined, if the grayscale of the next frame satisfies the target grayscale, X-ray imaging may be performed with the target performing parameters of the next frame, and if not, the target performing parameters may be updated based on closed-loop control.

In this way, the equivalent phantom thickness may be updated based on the correspondence to shorten the ABS stabilization time; and the stability of the brightness adjustment is taken into account by the subsequent closed-loop control.

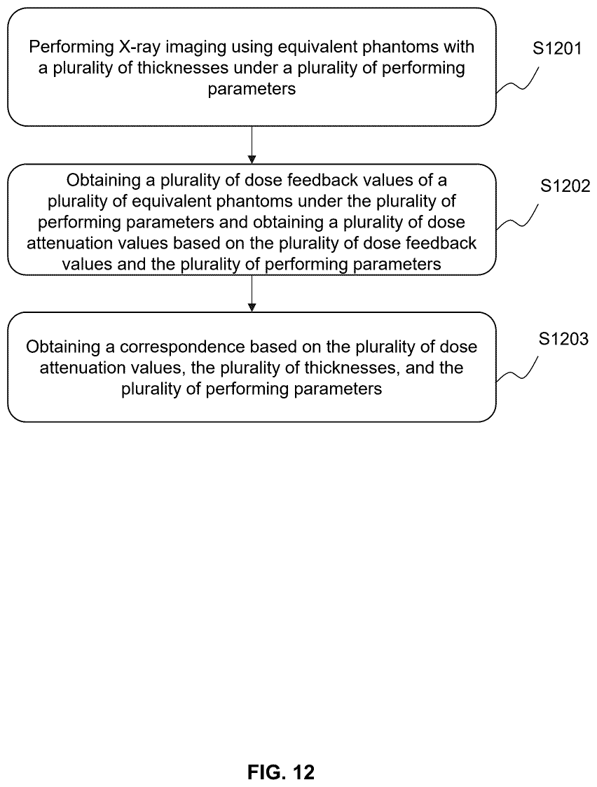

FIG. 12 is a flowchart illustrating another exemplary control process for a system for X-ray imaging according to some embodiments of the present disclosure. As shown in FIG. 12, the method may include obtaining a correspondence.

In operation S1201, X-ray imaging may be performed using equivalent phantoms with a plurality of thicknesses under a plurality of performing parameters.

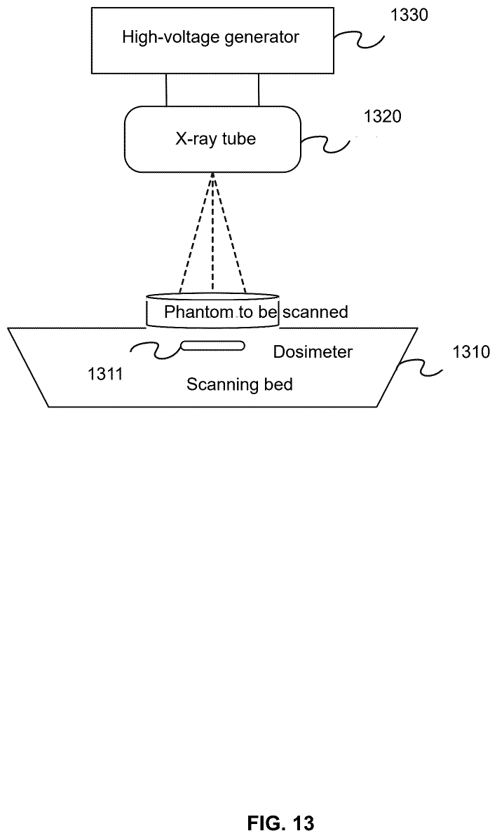

FIG. 13 is a flowchart illustrating another exemplary control process for a system for X-ray imaging according to some embodiments of the present disclosure. Operation S1301 may be implemented using a test platform as shown in FIG. 13. The test platform may include a scanning bed 1310, a dosimeter 1311 disposed on the scanning bed 1310, an X-ray tube 1320 disposed above the scanning bed 1310, and a high voltage generator 1330 electrically connected to the X-ray tube 1320.

The scanning bed 1310 may be used to carry an equivalent phantom that simulates an object. The dosimeter 1311 may be used to receive X-rays that pass through the equivalent phantom, thereby obtaining a dose feedback value. The high voltage generator 1330 may control an output of the X-rays from the X-ray tube 1320 to irradiate the equivalent phantom.

In operation S1202: a plurality of dose feedback values of a plurality of equivalent phantoms under a plurality of performing parameters may be obtained, and a plurality of dose attenuation values may be obtained based on the plurality of dose feedback values and the plurality of performing parameters.

The dose feedback value may be data directly obtained by the dosimeter 1311, and the dose attenuation value may be obtained through the dose feedback value, a performing time, and a tube current. Specifically, the dose attenuation value may be a ratio of the dose feedback value to a product of the performing time and tube current. The dose attenuation value obtained by operation S1202 removes effects of the tube current and the performing time, thereby reflecting a relationship between the dose attenuation value and the tube voltage.

In operation S1203, a correspondence may be obtained based on the plurality of dose attenuation values, the equivalent phantom thickness, and the plurality of performing parameters.

Optionally, the dose attenuation values are normalized. For example, a certain performing parameter (e.g., 80 kv) may be selected as a reference, and the dose attenuation values corresponding to the other performing parameters may be divided by a dose attenuation value corresponding to 80 kv under a same equivalent phantom thickness to obtain the dose attenuation values in the correspondence. The correspondence obtained in this way is suitable for updating the equivalent phantom thickness based on a combination of the current frame and the previous frame. Because of a normalization process, it is easier to update the equivalent phantom thickness based on the correspondence based on the grayscales and the performing parameters of the current frame and the previous frame.

Optionally, the correspondence may be established directly based on the dose attenuation values, the equivalent phantom thickness, and the performing parameters, i.e., the correspondence may be established without normalization. The correspondence obtained in this way is suitable for directly determining the equivalent phantom thickness based on the grayscale and the current performing parameters of the current frame, which may improve the efficiency of updating the equivalent phantom thickness.

In summary, the control process for the system for X-ray imaging provided by the embodiments of the present disclosure may perform an equivalent phantom update by means of a pre-obtained correspondence, which enables to a rapid determination of suitable performing parameters and a short stabilization time of the ABS. At the same time, a closed-loop control manner is adopted to take into account the stability of ABS adjustment, thereby optimizing user experience.

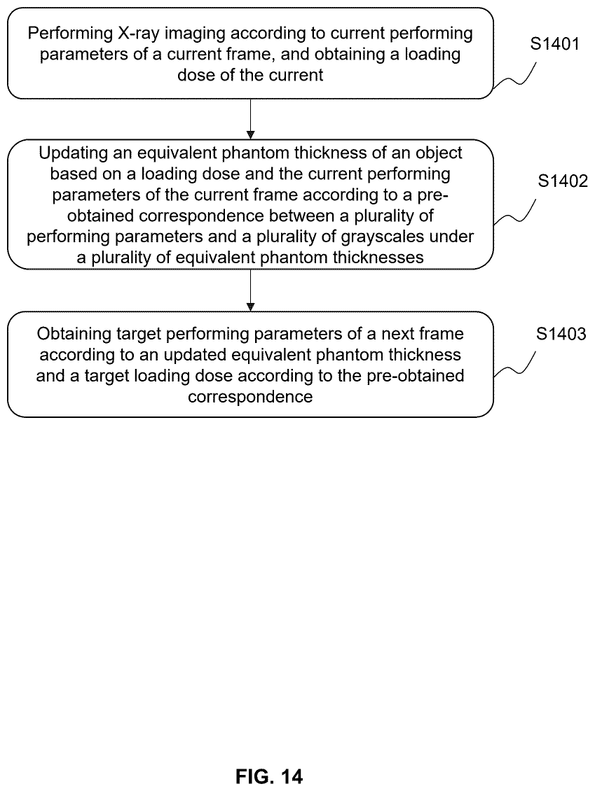

Some embodiments of the present disclosure may provide a control method for a system for X-ray imaging. FIG. 14 is a flowchart illustrating another exemplary control process for a system for X-ray imaging according to some embodiments of the present disclosure. As shown in FIG. 14, the method may include following operations.

In operation S1401, X-ray imaging may be performed according to current performing parameters of a current frame, and a loading dose of the current frame may be obtained.

In operation S1402, an equivalent phantom thickness of an object may be updated based on the loading dose and the current performing parameters of the current frame according to a pre-obtained correspondence between a plurality of performing parameters and a plurality of grayscales under a plurality of equivalent phantom thicknesses.

In operation S1403, target performing parameters of a next frame may be obtained according to an updated equivalent phantom thickness and a target loading dose according to the pre-obtained correspondence.

According to the process provided in this embodiment, the equivalent phantom thickness may be updated using the correspondence based on the loading dose and the current performing parameters of the current frame. Thereby, the equivalent phantom thickness may be close to an actual situation of the current object, so as to increase an updating rate of subsequent performing parameters, shorten a stabilization time of ABS, and solve technical defects in the related technology.

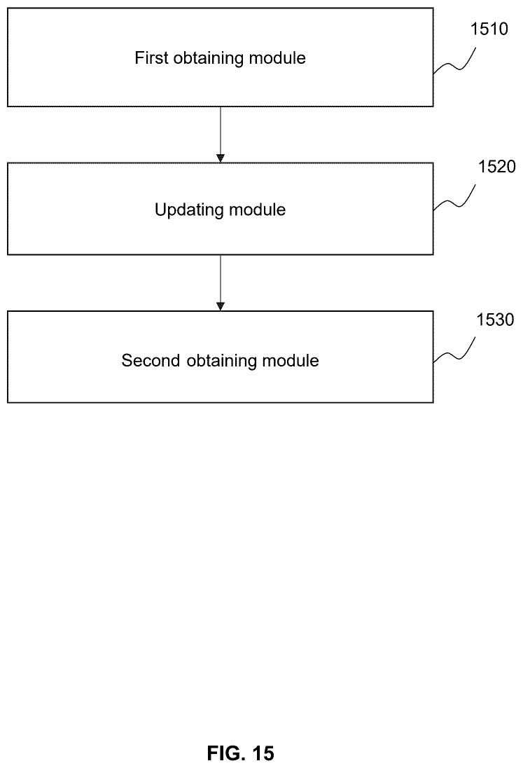

FIG. 15 is a block diagram illustrating a control device for a system for X-ray imaging according to some embodiments of the present disclosure. As shown in FIG. 15, the control device for the system for X-ray imaging may include: a first obtaining module 1510, an updating module 1520, and a second obtaining module 1530.

The first obtaining module 1510 may be configured to perform X-ray imaging according to current performing parameters of a current frame and obtain a grayscale of the current frame.

The updating module 1520 may be configured to, based on the grayscale and the current performing parameters of the current frame, update an equivalent phantom thickness of an object according to a pre-obtained correspondence between a plurality of performing parameters and a plurality of dose attenuation values under a plurality of equivalent phantom thicknesses.

The second obtaining module 1530 may be configured to obtain target performing parameters of a next frame based on an updated equivalent phantom thickness and a target grayscale according to the pre-obtained correspondence.

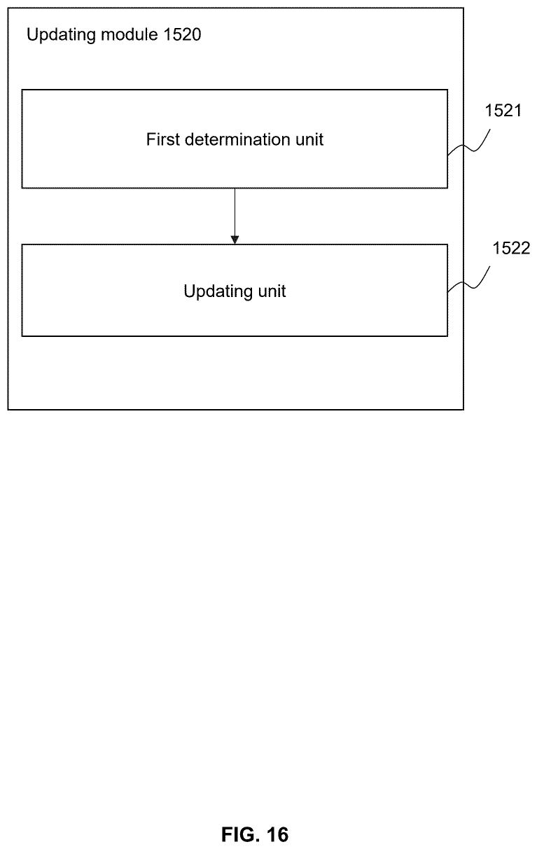

FIG. 16 is a block diagram illustrating the updating module 1520 according to some embodiments of the present disclosure. As shown in FIG. 16, the updating module 1520 may include: a first determination unit 1521 and an updating unit 1522.

The first determination unit 1521 may be configured to determine a first modulation strategy based on a grayscale of a current frame and a grayscale of a previous frame.

The updating unit 1522 may be configured to update an equivalent phantom thickness according to a correspondence based on the first modulation strategy, current performing parameters of the current frame, and previous performing parameters of the previous frame.

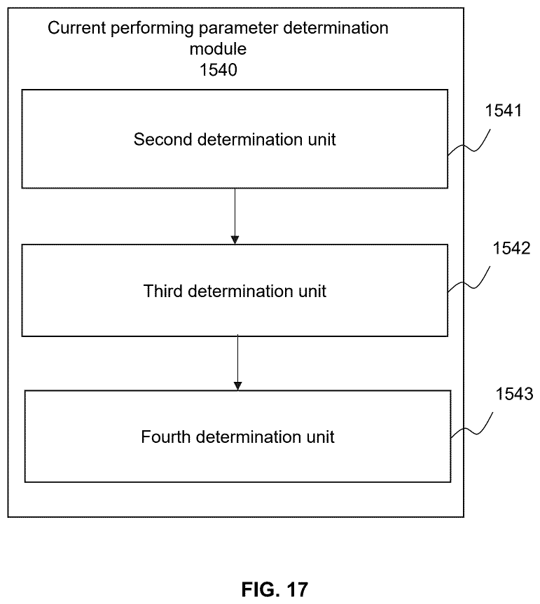

FIG. 17 is a block diagram illustrating another control device for a system for X-ray imaging according to some embodiments of the present disclosure. The control device may further include a current performing parameter determination module 1540. The current performing parameter determination module 1540 may specifically include a second determination unit 1541, a third determination unit 1542, and a fourth determination unit 1543.

The second determination unit 1541 is configured to determine a first dose attenuation value based on previous performing parameters of a previous frame and a preset equivalent phantom thickness according to a correspondence.

The third determination unit 1542 is configured to determine a second modulation strategy based on a grayscale of a previous frame and a target grayscale.

The fourth determination unit 1543 is configured to determine current performing parameters of a current frame according to the correspondence based on the first dose attenuation value and the first modulation strategy.

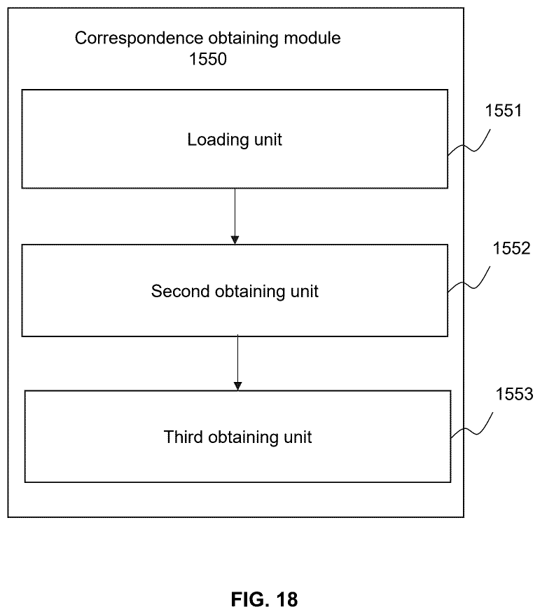

FIG. 18 is a block diagram illustrating another control device for a system for X-ray imaging according to some embodiments of the present disclosure. As shown in FIG. 18, the control device may further include a correspondence obtaining module 1550. The correspondence obtaining module 1550 may specifically include a loading unit 1551, a second obtaining unit 1552, a third obtaining unit 1553.

The loading unit 1551 is configured to perform X-ray imaging under a plurality of performing parameters using a plurality of equivalent phantom thicknesses.

The second obtaining unit 1552 is configured to obtain a plurality of dose feedback values of a plurality of equivalent phantoms under the plurality of performing parameters and obtain a plurality of dose attenuation values based on the dose feedback values and the performing parameters.

The third obtaining unit 1553 is configured to obtain a correspondence based on the dose attenuation values, the equivalent phantom thicknesses, and the performing parameters.

In some embodiments, the control device may further include a first judgment module. The first judgment module may be configured to determine whether a grayscale of the current frame is within a target grayscale, if not, to determine whether the grayscale of the current frame is within a closed-loop control grayscale range, and if not, to perform an equivalent phantom update operation.

In some embodiments, the control device may further include a second judgment module. The second judgment module may be configured to determine whether the grayscale of the current frame is within the target grayscale, if not, to determine whether the grayscale of the current frame is within the closed-loop control grayscale range, and if not, to perform a current performing parameter obtaining operation.

In summary, the control device for the system for X-ray imaging provided in the embodiments of the present disclosure may update the equivalent phantom by means of the pre-obtained correspondence, which enables to a rapid determination of a suitable performing parameter and a short stabilization time of the ABS. At the same time, a closed-loop control manner is adopted to take into account the stability of ABS adjustment, thereby optimizing user experience.

Some embodiments of the present disclosure may further provide a system for X-ray scanning, which includes the control device for the system for X-ray imaging provided in the above embodiments.

In summary, the system for X-ray scanning provided by the embodiments of the present disclosure may update the equivalent phantom by means of the pre-obtained correspondence, which enables a rapid determination of suitable performing parameters and a short stabilization time of the ABS. At the same time, a closed-loop control manner is adopted to take into account the stability of ABS adjustment, thereby optimizing user experience.

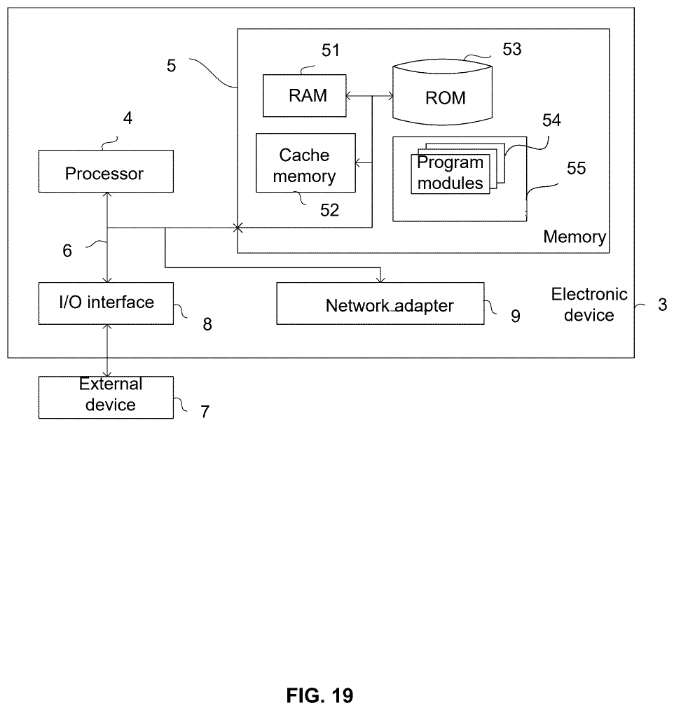

FIG. 19 is a schematic diagram illustrating a structure of an electronic device according to some embodiments of the present disclosure. The electronic device may include at least one processor and a memory communicatively connected to the at least one processor. The memory stores a computer program runnable by the at least one processor, the computer program may be executed by the at least one processor to enable the at least one processor to perform the control method for the system for X-ray imaging as shown in some embodiments of the present disclosure. An electronic device 3 shown in FIG. 19 is merely an example and should not impose any limitations on the functionality and scope of use of embodiments of the present disclosure.

Components of the electronic device 3 may include, but are not limited to, at least one processor 4, at least one memory 5, and a bus 6 connecting different system components including the memory 5 and the processor 4.

The bus 6 may consist of a data bus, an address bus, and a control bus.

The memory 5 may include a volatile memory, e.g., a random access memory (RAM) 51 and/or a cache memory 52, and may further include a read-only memory (ROM) 53.

The memory 5 may also include a program or utility 55 having a set (at least one) of program modules 54, the program modules 54 may include, but are not limited to: an operating system, one or more applications, other program modules, and program data. Each or a certain combination of the operating system, the one or more applications, the other program modules, and the program data may include an implementation of a network environment.

The processor 4 may perform various functional applications and data processing (e.g., the above-described control method for the system for X-ray imaging) by running one or more computer programs stored in the memory 5.

The electronic device 3 may also communicate with one or more external devices 7 (e.g., keyboards, pointing devices, etc.). The communication may be carried out via an input/output (I/O) interface 8. Furthermore, the electronic device 3 may also communicate with one or more networks (e.g., a local area network (LAN), a wide area network (WAN), and/or a public network, such as the Internet) via a network adapter 9. As shown in FIG. 19, the network adapter 9 may communicate with other modules of the electronic device 3 via the bus 6. It should be appreciated that, although not shown in FIG. 19, other hardware and/or software modules may be used in conjunction with the electronic device 3, e.g., microcode, a device driver, a redundant processor, an external disk drive arras, a RAID (disk array) system, a tape drive, a data backup storage system, or the like, or any combination thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or collocation of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer-readable program code embodied thereon.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required characteristics of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

1. A system for X-ray imaging, comprising:
a storage device storing a set of instructions; and
at least one processor in communication with the storage device, wherein when executing the set of instructions, the at least one processor is directed to cause the system to perform operations including:
obtaining a grayscale of a current frame of an object, wherein the current frame is obtained by performing X-ray imaging on the object based on current performing parameters;
determining an updated equivalent phantom thickness of the object based on the grayscale of the current frame, the current performing parameters, and a pre-stored correspondence, wherein the pre-stored correspondence reflects dose values corresponding to a plurality of equivalent phantom thicknesses and a plurality of performing parameters; and
determining target performing parameters of a next frame of the object based on the updated equivalent phantom thickness, a target grayscale, and the pre-stored correspondence,
wherein the target performing parameters include a tube voltage, a tube current, a performing time of an X-ray tube, and the tube voltage, the tube current, the performing time satisfy a preset relationship, which be specified by an ABS brightness adjustment curve.

2. The system of claim 1, wherein the pre-stored correspondence includes the dose values corresponding to the plurality of equivalent phantom thicknesses and the plurality of performing parameters.

3. The system of claim 1, wherein the pre-stored correspondence includes normalized values of dose attenuations corresponding to the plurality of equivalent phantom thicknesses and the plurality of performing parameters.

4. The system of claim 1, wherein the determining the updated equivalent phantom thickness of the object based on the grayscale of the current frame includes:
obtaining a grayscale of a previous frame of the current frame, the previous frame being obtained by performing X-ray imaging on the object based on previous performing parameters; and
determining the updated equivalent phantom thickness of the object based on the grayscale of the current frame, the grayscale of the previous frame, the current performing parameters, the previous performing parameters, and the pre-stored correspondence.

5. The system of claim 4, wherein the determining the updated equivalent phantom thickness of the object based on the grayscale of the current frame, the grayscale of the previous frame, the current performing parameters, the previous performing parameters, and the pre-stored correspondence includes:
determining a ratio of the grayscale of the current frame to the grayscale of the previous frame; and
determining the updated equivalent phantom thickness of the object based on the ratio of the grayscale of the current frame to the grayscale of the previous frame, the current performing parameters, the previous performing parameters, and the pre-stored correspondence.

6. The system of claim 1, wherein the determining the target performing parameters of the next frame of the object based on the updated equivalent phantom thickness, the target grayscale, and the pre-stored correspondence includes:
determining whether the grayscale of the current frame is within a target grayscale value interval; and
in response to determining that the grayscale of the current frame is within the target grayscale value interval, designating the current performing parameters as the target performing parameters of the next frame.

7. The system of claim 6, wherein the operations further include:
in response to determining that the grayscale of the current frame is out of the target grayscale value interval, determining whether the grayscale of the current frame is within a closed-loop control grayscale range; and
in response to determining that the grayscale of the current frame is not within the closed-loop control grayscale range, determining performing parameters that satisfy the target grayscale as the target performing parameters based on the grayscale of the current frame, the current performing parameters, the updated equivalent phantom thickness, and the pre-stored correspondence.

8. (canceled)

9. The system of claim 1, wherein the pre-stored correspondence is obtained by:
obtaining a plurality of dose values of the plurality of equivalent phantom thicknesses under the plurality of performing parameters, the dose values being obtained by performing X-ray imaging on a plurality of equivalent phantoms with a plurality of thicknesses under the plurality of performing parameters; and
determining the pre-stored correspondence based on the plurality of dose values, the plurality of equivalent phantom thicknesses, and the plurality of performing parameters.

10. The system of claim 1, wherein the pre-stored correspondence is obtained by:
obtaining a plurality of dose values of the plurality of equivalent phantom thicknesses under the plurality of performing parameters, the dose values being obtained by performing X-ray imaging on a plurality of equivalent phantoms with a plurality of thicknesses under the plurality of performing parameters;
obtaining a plurality of normalized values of dose attenuations based on the plurality of dose values and the plurality of performing parameters; and
determining the pre-stored correspondence based on the plurality of normalized values of the dose attenuations, the plurality of equivalent phantom thicknesses, and the plurality of performing parameters.

11. (canceled)

12. A method for X-ray imaging, comprising:
obtaining a grayscale of a current frame of an object, wherein the current frame is obtained by performing X-ray imaging on the object based on a current performing parameters;
determining an updated equivalent phantom thickness of the object based on the grayscale of the current frame, the current performing parameters, and a pre-stored correspondence, wherein the pre-stored correspondence reflects dose values corresponding to a plurality of equivalent phantom thicknesses and a plurality of performing parameters; and
determining target performing parameters of a next frame of the object based on the updated equivalent phantom thickness, a target grayscale, and the pre-stored correspondence,
wherein the target performing parameters include a tube voltage, a tube current, a performing time of an X-ray tube, and the tube voltage, the tube current, the performing time satisfy a preset relationship, which be specified by an ABS brightness adjustment curve.

13. The method of claim 12, wherein the pre-stored correspondence includes the dose values corresponding to the plurality of equivalent phantom thicknesses and the plurality of performing parameters.

14. The method of claim 12, wherein the pre-stored correspondence includes normalized values of dose attenuations corresponding to the plurality of equivalent phantom thicknesses and the plurality of performing parameters.

15. The method of claim 12, wherein the determining the updated equivalent phantom thickness of the object based on the grayscale of the current frame includes:
obtaining a grayscale of a previous frame of the current frame, the previous frame being obtained by performing X-ray imaging on the object based on previous performing parameters; and
determining the updated equivalent phantom thickness of the object based on the grayscale of the current frame, the grayscale of the previous frame, the current performing parameters, the previous performing parameters, and the pre-stored correspondence.

16. (canceled)

17. The method of claim 12, wherein the determining the target performing parameters of the next frame of the object based on the updated equivalent phantom thickness, the target grayscale, and the pre-stored correspondence includes:
determining whether the grayscale of the current frame is within a target grayscale value interval; and
in response to determining that the grayscale of the current frame is within the target grayscale value interval, designating the current performing parameters as the target performing parameters of the next frame.

18. The method of claim 17, further comprising:
in response to determining that the grayscale of the current frame is out of the target grayscale value interval, determining whether the grayscale of the current frame is within a closed-loop control grayscale range; and
in response to determining that the grayscale of the current frame is not within the closed-loop control grayscale range, determining performing parameters that satisfy the target grayscale as the target performing parameters based on the grayscale of the current frame, the current performing parameters, the updated equivalent phantom thickness, and the pre-stored correspondence.

19. (canceled)

20. The method of claim 12, wherein the pre-stored correspondence is obtained by:
obtaining a plurality of dose values of the plurality of equivalent phantom thicknesses under the plurality of performing parameters, the dose values being obtained by performing X-ray imaging on a plurality of equivalent phantoms with a plurality of thicknesses under the plurality of performing parameters; and
determining the pre-stored correspondence based on the plurality of dose values, the plurality of equivalent phantom thicknesses, and the plurality of performing parameters.

21. The method of claim 12, wherein the pre-stored correspondence is obtained by:
obtaining a plurality of dose values of the plurality of equivalent phantom thicknesses under the plurality of performing parameters, the dose values being obtained by performing X-ray imaging on a plurality of equivalent phantoms with a plurality of thicknesses under the plurality of performing parameters;
obtaining a plurality of normalized values of dose attenuations based on the plurality of dose values and the plurality of performing parameters; and
determining the pre-stored correspondence based on the plurality of normalized values of the dose attenuations, the plurality of equivalent phantom thicknesses, and the plurality of performing parameters.

22. (canceled)

23. A non-transitory computer-readable storage medium storing computer instructions, wherein after reading the computer instructions in the storage medium, a computer executes the method for X-ray imaging of claim 12.

24. The system of claim 1, wherein the target performing parameters are selected from a plurality of working performing parameter corresponding to a plurality of working points in the ABS brightness adjustment curve.

25. The system of claim 1, wherein the determining target performing parameters of a next frame of the object based on the updated equivalent phantom thickness, a target grayscale, and the pre-stored correspondence includes:
determining the tube voltage based on updated equivalent phantom thickness, target grayscale and the pre-stored correspondence, and
determining the tube current and the performing time corresponding to the tube voltage based on the ABS brightness adjustment curve.
